(19) United States
(12) Patent Application Publication
(10) Pub. No.: US 2011/0209701 A1
(43) Pub. Date: Sep. 1, 2011

(54) Nose pad cushion and applicator for respiratory mask

(76) Inventors: Laura Derringer, Richmond, KY (US); Crystal Mitcheltree, Paris, KY (US)

(21) Appl. No.: 12/660,589

(22) Filed: Mar. 1, 2010

(51) Int. Cl. A61M 31/00; A61M 16/06

(52) U.S. Cl. 128/202.17; 128/206.25

(57) ABSTRACT

A removable nose pad cushion adherable to a medical face mask and more particularly to a positive pressure ventilation mask approximately where the mask rests on the upper nose area of a wearer's face providing a cushioning seal between the mask and wearer's nose. The nose pad cushion can be impregnated with medication to be adsorbed by the skin of the patient. The surface of the nose pad cushion which contacts the skin of the user is soft and pliable and selected to removably adhere only to dry skin and is incapable of sticking to a moist wound bed or sores formed on the bridge of the nose. The area covered by the cushion primarily covers the upper nose area but could cover the entire interface between the mask and the face of a user. The cushion can be pre-shaped to be applied to the desired area of the mask. The adhesive which holds the cushion to the mask is of a type such that the cushion can be removed cleanly and replaced with another cushion periodically.

### TECHNICAL FIELD

The present invention relates to the field of face masks used in the medical field for purposes of oxygen supply, and the like, and more recently for use with CPAP (continuous positive airway pressure) devices. In particular, the present invention is a cushion to be applied to a selected area on the face mask or on a cushion of a face mask covering a selected are of the face to increase the comfort to the face mask user and to prevent or to help treat sores and sore spots at the interface between a face mask and a user's face, especially on the upper nose area.

### BACKGROUND OF THE INVENTION

Positive pressure face masks are often used in the treatment of respiratory conditions and/or sleep disorders for delivering a positive air pressure flow of breathable air to assist in patient respiration. The masks usually are connected to an air supply through an air supply line connected to a mask held securely to the wearer's face or at least a portion of the face such as the nose. The walls of the mask body are usually formed of a semi rigid resilient flexible polymer and/or elastomeric material such as a silicon material which holds its shape. A cushion or other flexible seal means and/or a liner is often used with or formed integrally with the mask body and frame to conform to the contours of the wearer's face. The mask is typically secured to the wearer's head over the face by adjustable straps which can pulled tight enough to form a air tight seal with the wearer's face and the mask. While the present invention is utilized with air it is applicable to applications involving the use of oxygen or other gases.

A continuous positive airway pressure (CPAP) machine was initially used mainly by patients for the treatment of sleep apnea at home, but now is in widespread use across intensive care units as a form of ventilation. Obstructive sleep apnea occurs when the upper airway becomes narrow as the muscles relax naturally during sleep. This reduces oxygen in the blood and causes arousal from sleep. The CPAP machine stops this phenomenon by delivering a stream of compressed air via a hose to a nasal pillow, nose mask or full-face mask, splinting the airway (keeping it open under air pressure) so that unobstructed breathing becomes possible, reducing and/or preventing apneas and hypopneas. It is the air pressure, and not the movement of the air, that prevents the apneas. When the machine is turned on, but prior to the mask being placed on the head, a flow of air comes through the mask. After the mask is placed on the head, it is sealed to the face and the air stops flowing. At this point, it is only the air pressure that accomplishes the desired result. This has the additional benefit of reducing or eliminating the extremely loud snoring that sometimes accompanies sleep apnea.

The CPAP machine blows air at a prescribed pressure (also called the titrated pressure). The necessary pressure is usually determined by a sleep physician after review of a study supervised by a sleep technician during an overnight study polysomnography) in a sleep laboratory. The titrated pressure is the pressure of air at which most (if not all) apneas and hypopneas have been prevented, and it is usually measured in centimeters of water (cm H₂O). The pressure required by most patients with sleep apnea ranges between 6 and 14 cm H2O. A typical CPAP machine can deliver pressures between 4 and 20 cm H₂O. More specialized units can deliver pressures up to 25 or 30 cm H₂O. CPAP treatment can be highly effective in treatment of obstructive sleep apnea.

APAP or AutoPAP or AutoCPAP (Automatic Positive Airway Pressure) automatically titrates, or tunes, the amount of pressure delivered to the patient to the minimum required to maintain an unobstructed airway on a breath-by-breath basis by measuring the resistance in the patient's breathing, thereby giving the patient the precise pressure required at a given moment and avoiding the compromise of fixed pressure.

VPAP or BiPAP (Variable/Bilevel Positive Airway Pressure) provides two levels of pressure: Inspiratory Positive Airway Pressure (IPAP) and a lower Expiratory Positive Airway Pressure (EPAP) for easier exhalation. (Some people use the term BPAP to parallel the terms APAP and CPAP.)

The mask can be a full face mask or as shown in the drawings a nasal mask. A typical system would include a flow generator (PAP machine) provides the airflow, hose connecting the flow generator to the interface comprising a nasal or full face mask which provides the connection to the user's airway. Flexible chin straps may be used to help the patient not breathe through the mouth (full-face masks avoid this problem), thereby keeping a closed pressure system. The straps are elastic enough that the patient can easily open his mouth if he feels that he needs to. Modern straps use a quick-clip instant fit. Velcro-type adjustments allow quick sizing, before or after the machine is turned on.

The mask cushion of the instant invention is adaptable to be used providing additional cushioning on a variety of face masks. For example, a typical face mask is shown in U.S. Pat. No. 7,069,933 by Kwok et al. entitled “Breathing Mask and Mask Cushion Therefore” which issued on Jul. 4, 2006 and is incorporated by reference herein. The present invention is applicable to other NPPV mask companies such as Fisher Paykel, Nelcor Puritan Bennet, Respironics, etc.

As described in detail in U.S. Pat. No. 7,069,933, these mask use a substantially triangularly shaped frame having a scalloped edge whereby a full face cushion is affixed to the mask body. The cushion includes a triangular shaped aperture into which the wearer's nose is received. The cushion is spaced away from the rim of the frame and its outer surface is substantially shaped the same as the rim of the frame. Notches are formed in the cushion to receive the bridge of the wearer's nose. The wearer's nose and lips are received through the aperture into a chamber within the mask body. The cushion forms a seal with the wearer's nose and a portion of the wearer's face in the region between the bottom lip and the chin, and around the sides and over the bridge of the nose. The cushion forms a seal the along the nose and face.

Various materials such as cotton, elastomers, foams, gels, moleskin, have been used to cushion face masks. U.S. Patent Application Publication No. 20050199239 by Lang et al. for MASK CUSHIONING AND FOREHEAD PAD FOR A RESPIRATORY MASK, RESPIRATORY MASK IN ADDITION TO A MOULD AND METHOD FOR THEIR PRODUCTION published on Sep. 15, 2005, teaches a face mask with a molded in elastomeric cushion located at the desired interface between the mask and the face of the user. U.S. Patent Application Publication No. 20090139525 by Schirm for COMFORT LINER FOR PRESSURE FACE MASKS AND NASAL MASKS published on Jun. 4, 2009 teaches a cushioning liner preferably made from Dr. Scholl's MOLESKIN which is removably fastened to the surface of the face mask which contacts the face of the user.

Lang and Schism and others teach the use of a conventional cushioning material used with or integrally secured with to the mask to soften the load which the mask applies to the face. Most cushion materials used in conventional masks address the problem of holding the mask in position in order to prevent the wearer from dislodging the mask in sleep or breaking the air tight seal.

However, common problems occur at the upper nose area when face masks are worn on a long term basis. Sometimes the mask is applied so that it puts excess pressure to the face of the user causing skin irritation and discomfort to the wearer. The problem often is not discovered until facial soreness and ulceration occurs which is especially a problem in the area around the bridge of the wearer's nose. If there are minor lesions or sores in that area due to a prior injury or application of the mask causes the sores, continued use of the conventional cushioning material exacerbates the problem by sticking to the sore spots and tearing or irritating them.

### SUMMARY OF THE INVENTION

As shown in the figures, one preferred embodiment utilizes a ventilation mask typically comprising a total nose mask or total face mask wherein the mask fits over the nose and mouth whereby pressure and suction of the air controlled by the ventilation unit. Conventional mask include a soft flexible plastic, rubber, or foam backing interface which rests upon the skin of the user; however, wearing of the mask for an extended length of time still leads to the wearer developing sores on the bridge of the nose similar to bed sores because of the small amount of fatty tissue which covers the bridge of the nose.

The present invention provides for a nose pad cushion for attachment to a mask. The nose pad cushion includes a layer of cushioning material permanently or removably attached to the interface of the mask by an adhesive, plastic welding, or other means of attachment such as an adhesive strip forming an air tight seal therewith. The cushion can even be molded integrally with the mask, but for long term use it is advantageous to provide the nose pad cushion as a removable disposable cushion or a reattach able cushion which can be used for application of medicine in the form of liquids, gels, slurries, paste, salve and the like. The cushion can include an outer surface including pores, micro grooves, pits, or other textured surface means for holding a liquid, paste, powder, gel, salve, or other form of a medical product impregnated thereon between the user's skin and the cushion surface. Such gels or other viscous materials can not be readily used with the cushions which come with the mask because contamination or cessation of treatment would require disposal of the entire mask.

The cushion may be designed to fit around the periphery of the mask or formed to cover the bridge of the nose and resting on the cheeks so long as an air tight seal is maintained between the user's face and the mask. The cushion can also be formed in a smaller triangle to fit over the user's nose and rest upon the users It should be noted that an air tight seal can be further defined as a seal sufficient to provide the patient with sufficient air under normal ventilation machine operating conditions. Often such machines are used to aid patients in breathing and it is not necessary to maintain a perfect seal under all conditions such as when a patient is unable to breath on their own so long as positive air pressure is achieved to aid the patient with respiration.

It is an object of the present invention to provide a nose pad cushion to replace to a face mask, or attach to an existing face mask cushion for additional support for use with a positive pressure ventilation mask wherein the ask above and around the sides of the nose are designed to provide sufficient sealing for against positive air pressure for ventilation yet prevent sores or sore spots on the upper area of the nose after extended wearing of the mask. The cushioning material may be molded into the mask or may be adhered to a mask with an adhesive, adhesive strip, coextrusion or the like. The suctioning may contain one or more pores for administering an antibiotic or other drug and/or be impregnated with antibiotics, other medications, or even time release medications which are secreted onto the desired area of the mask cushion around the wearer's nose. In addition to medicinal applications, the outer surface of the nose pad cushion may include or substitute a gel for promoting an air tight seal or lubrication, fibron glue, or other viscous substance to prevent adhesions and irritation of the patient's skin by the mask cushion and to promote healing.

The present invention solves the problem of irritation caused by a face mask, especially around the upper nose area by providing a cushion that can be applied to the face mask prior to use which will not stick to the moist wound bed present when small lesions or sores are present on the skin. Further, the impregnation of the mask cushion with a healing promoter such as the fibrin glue or fibrin sealant prevents damage to the skin, increases comfort, and act as a membrane barrier or lubricant optionally impregnated with medication which will transfer out to the skin to sooth and or help heal any sores or lesions which may be present.

It is an object of this invention to provide a cushioning pad which will not cause irritation to the skin when wearing a medical face mask.

It is an object of this invention to provide a cushioning pad which will not stick to sores or lesions on the facial skin, especially in the upper nose area, caused by long term use of a face mask as would occur, for example, with CPAP users.

It is an object of this invention to provide a cushioning pad which will be provided with an adhesive which would hold the cushion in place on the face mask but could easily by removed and replaced with a fresh cushion.

It is an object of the present invention to provide a cushioning pad which can be changed to extend the life of the mask.

It is an object of the present invention to provide a cushioning pad which is disposable.

It is an object of the present invention to provide a mask cushion pad which seals the mask to the patient's skin, but is removable, peelable, and breaks off withing the plane minimizing irrigation to the patient's skin.

It is an object of the present invention to provide an adhesive comprising a cryoprecipate fibrin glue to removably adhere the mask cushion to the skin of the patient having a sore or irritated skin to reduce inflammation and promote healing of the skin over the bone at the bridge of the nose and enhance cellular regeneration thereon.

It is an object of the present invention to provide to provide a mask with a cushion pad having an interior portion composed of a foam material and an exterior surface covered by a plastic film.

It is an object of the present invention to provide cover a mask with a cushion pad having an interior portion composed of a foam material and an exterior surface covered by a textured plastic film.

It is an object of the present invention to provide cover a mask with a cushion pad having an interior portion composed of a foam material, a plastic film covering the foam material for adhering to the patient's skin, and a plastic film covering and extending beyond the exterior surface of the foam cushion and cover a portion of the mask interface for adhering thereto.

It is an object of this invention to provide a cushioning pad which will be provided with an adhesive, on the side of the cushion which contacts the face, using the SAFETAC technology, which prevents a bandage from sticking to the moist wound bed will stick to surrounding dry skin.

It is an object of the present invention to provide an adherence to dry skin of 0.2-3.0 N/25 nm and preferably 1 to 2.5 N/25 nm and incorporates by reference U.S. Patent Publication 20080097361 published on Apr. 24, 2008.

### BRIEF DESCRIPTION OF THE DRAWINGS

A better understanding of the present invention will be had upon reference to the following description in conjunction with the accompanying drawings in which like numerals refer to like parts throughout the views wherein:

FIG. 1 is a oblique view of a typical face mask;

FIG. 2 is and oblique view of a cushioning pad to be applied to the nose area of a typical face mask;

FIG. 3 is and oblique view of an alternate embodiment which covers more than just the upper nose area;

FIG. 4 is an isometric view of a cushioning nose pad showing a split foam center covered by a plastic film on both side to be coated on one side with an adhesive suitable to be removable from the patient's skin and the other side of the plastic film not shown to be covered with an adhesive for adhering the nose pad cushion to the interface of a ventilation mask;

FIG. 5 is a isometric view of the nose pad cushion shown in FIG. 4, wherein a outer covering constitutes a membrane or sheath extending over the cushion body to form a flap for adherence to the mask cushion body

### DESCRIPTION OF THE PREFERRED EMBODIMENTS

In accordance with the present invention, there is provided a cushioning pad to be applied to the portion of the surface a face mask which comes into contact with the skin of a user and especially that portion which contacts the area around and above the nose. Users who frequently use a face mask, e.g. CPAP users, commonly have irritation and even sores around and above the nose.

According to findings attributed to Molnlycke Health Care AB, Goteborg, Sweden, “When a wound is traumatized during removal of a dressing that has adhered to the wound, the patient frequently experiences pain. The pain produces a stress response that results in increased production of catecholamines and cortisol. These increased levels of cortisol suppress the immune response by decreasing the activity of white blood cells that have an important role in cleaning the wound. Over-production of cortisol has also been implicated in the decrease of collagen production, a vital component of granulation tissue. Cortisol increases catabolism, which leads to the breakdown of vital nutrients that are needed by the cells during wound healing. Cortisol also stimulates the production of catecholamines, including noradrenalin and adrenalin. An increase in catecholamines leads to vasoconstriction of small arterioles. This decreases the peripheral blood supply, resulting in the reduction of oxygen and nutrients being delivered to the wound, combined with a decreased resistance to infection. The increase in catecholamines also leads to an increase in epidermal hormones or chalones, which inhibit the mitosis and maturation of epithelial cells. (Diane Joseph, When healing is a pain. Nursing Times 1997; 93:16:85-6).

Often the use of positive pressure respirator masks causes the surrounding skin to become traumatized, flaky and dry. Excoriation and maceration by wound exudate is a common problem. Fragile skin adjacent to a wound may tear, while allergic reactions can occur with certain types of dressings and the tapes used to hold them in position such as contact dermatitis. Care of the surrounding skin is often overlooked and examination of the would or irritated or sore causes discomfort for the patient.

Traditional dressings incorporating gauze, paraffin-impregnated tulle dressings and VASELINE petrolatum based dressings, are frequently cited as causes of pain in conjunction with dressing changes. Gauze easily dries out and hardens when in place. In addition granulation tissue grow through the fabric, causing tissue trauma and considerable pain as it is removed. Removal of bandages having traditional adhesives are accompanied by large areas of the stratum corneum, that is to say the top layer of the skin, which gives rise to redness and, in certain cases, irritation of the skin.

A novel feature of the present invention includes an adhesive providing an undisturbed healing environment, adheres gently to dry tissue, but not to moist wound surfaces, molds to the skin's irregularities, covering more skin surface and spreading peel forces on removal to prevent skin stripping, and seals the wound margins and ensures exudate does not spread to the surrounding skin which minimizes maceration. An example of such an adhesive is sold under the trademark name of SAFETAC and is available from Molnlycke Health Care AB, Goteborg, Sweden. At least one type of adhesive comprises a soft silicone which conforms to the skin's irregularities while other adhesives stick to the uppermost surface of the epidermis. This conforming action is the key to why the adhesive is effective at minimizing pain and preventing maceration. Moreover, the adhesive does not adhere to the moist wound but adheres gently to the dry tissue surrounding same and promotes healing of wounds caused by friction, shear, or pressure such as typically experienced by users of a nasal mask or face mask.

One embodiment of the nose pad cushion of the present invention is also available as a soft conformable absorbent self-adhesive pad comprising, consisting essentially of, or consisting of at least one thin flexible absorbent pad of polyurethane foam sandwiched between at least one soft silicone foam layer which is atraumatic to the wound and surrounding skin upon removal to minimize maceration risk. The nose pad cushion is coated on one side with an adhesive which removably adheres the pad to the portion of the mask which typically rests upon the patients skin.

Another preferred embodiment of nose pad cushion of the present invention comprises, consists essentially of or consists of at least one soft thin flexible sheet of absorbent comprising a hydrophilic polyurethane foam of a selected thickness for example, about four millimeters thick sandwiched between at least one layer of soft silicone which may be a foam. In at least one preferred embodiment the polyurethane is a semi-permeable polyurethane film. One side of the soft silicone is bonded to a water vapor-permeable polyurethane film backing layer which is moisture proof and bacteria proof.

Another preferred embodiment of nose pad cushion of the present invention comprises, consists essentially of or consists of at least one soft thin flexible sheet of absorbent comprising a fibrous material such as a synthetic fiber and/or a cotton batting similar to quilt batting sandwiched between at least one soft thin flexible layer of soft silicone which may comprise a foam material. A absorbent material such as a gauze material covers the side of the soft silicone which contacts the skin. The gauze material may impregnated with medication cells, an antibiotic, salve, gel, bactericide, sterile solution, drugs, or other medium containing a form of medication to interface with the wearer's skin. A film of adhesive removably secures the soft silicone layer to the skin; however, a water vapor-permeable polyurethane film backing layer which is moisture proof and bacteria proof could be used to cover the silicone layer or the gauze layer adjacent the skin and/or the silicone layer adjacent the mask surface. The permeable polyurethane film can include grooves, pores, or other openings to allow for application of drugs or the other medications applied and held by the pad cushion to contact the skin treating same. An adhesive such as soft silicone which conforms to the skin's irregularities and/or fibrin glue is used to hold the nose pad in place on the skin, while a stronger removable adhesive would be used to attach the nose pad to the surface of the mask.

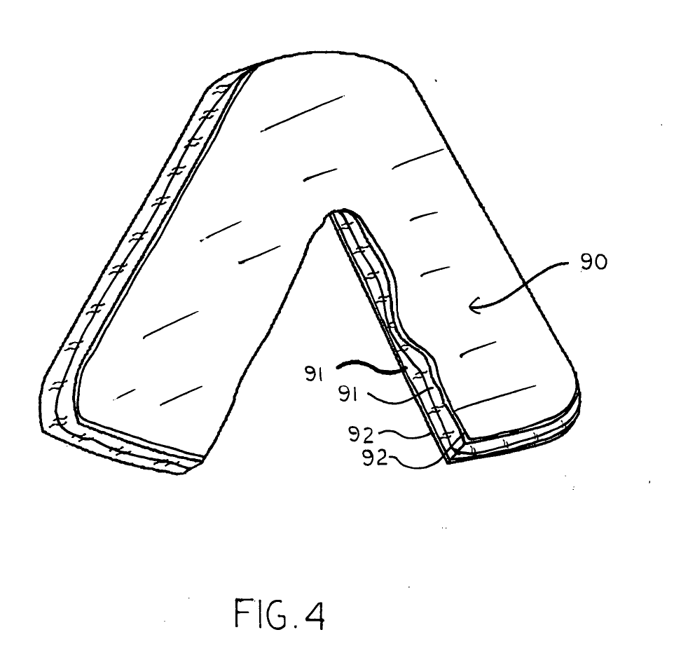

As shown in FIG. 4, the nose pad cushion 90 is folded over and the wound contact surface of the dressing is coated with a soft silicone adhesive layer which adheres to the peri-wound skin, retaining the dressing in position and providing a moist wound-healing environment. As shown, nose pad cushion 90 of the present invention comprises, consists essentially of or consists of at least one soft thin flexible sheet of absorbent comprising a fibrous material 91 such as a synthetic fiber and/or a cotton batting similar to quilt batting sandwiched between at least one soft thin flexible layer of soft silicone 92 which may comprise a foam material folded over. The surface of the nose pad cushion removably attachable to the mask or mask cushion is secured with a stronger removable adhesive. The wound contact surface of the dressing is protected with a plastic film, which must be removed before use.

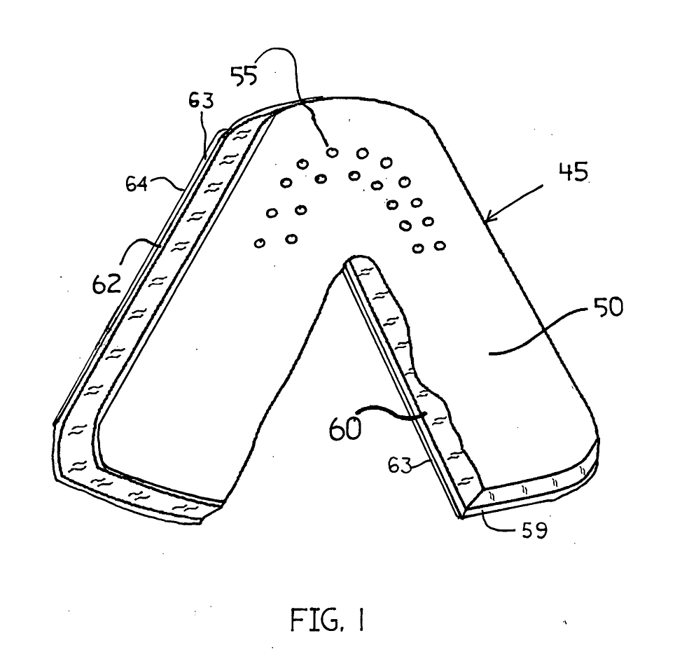

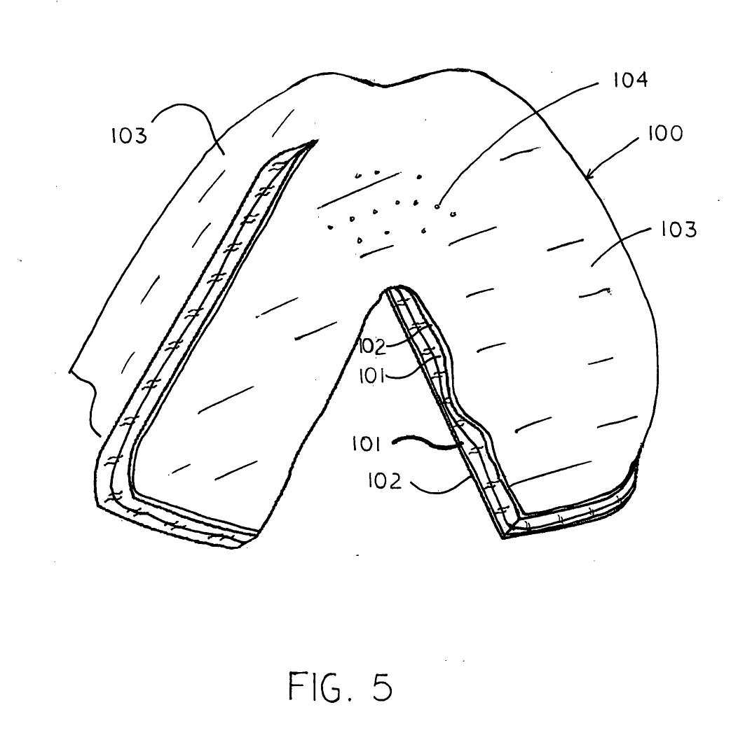

FIG. 5 shows a nose pad cushion 100 similar to that described in FIG. 1, 3, or 4 including a water vapor-permeable polyurethane film backing layer 103 which is moisture proof and bacteria proof could be used to cover the silicone layer 102 which covers the absorbent layer 101 and is folded over same. A plurality of pores 104 is formed in the polyurethane film layer 103. The film backing covering the nose pad can be covered with a removable adhesive and extended over the top of the nose mask cushion 40 wrapping partially around same to add in securing the nose pad cushion to the mask cushion.

The nose pad cushion can also be used to protect injured or fragile skin and be applied under compression bandaging and used in combination with other dressings such as hydrogels.

Another important feature of a preferred embodiment of the instant invention is the use of fibrin sealants as a type of surgical tissue adhesive derived from human and animal blood products. The ingredients in these sealants interact during application to form a stable clot composed of a blood protein called fibrin. Fibrin sealants are also called fibrin glues. all fibrin sealants used in the United States are made from blood plasma taken from carefully screened donors and rigorously tested to eliminate hepatitis viruses. Fibrin sealants have been used during surgery to control bleeding in the area where the surgeon is operating, to speed wound healing, to seal off hollow body organs or cover holes made by standard sutures, to provide slow-release delivery of medications to tissues exposed during surgery. Fibrin sealants stop bleeding and speed up the formation of a stable clot. They can be applied to very small blood vessels and to areas that are difficult to reach. Fibrin glues lower the risk of postoperative inflammation or infection, and are conveniently absorbed by the body during the healing process. They also reduce scar tissue. They are particularly useful for minimally invasive procedures and for treating patients with blood clotting disorders. Fibrin sealants have two major ingredients, a purified fibrinogen (a protein) and purified thrombin (an enzyme) derived from human or bovine (cattle) blood. Many sealants have two additional ingredients, human blood factor XIII and a substance called aprotinin, which is derived from cows' lungs. Factor XIII is a compound that strengthens blood clots by forming cross-links between strands of fibrin. Aprotinin is a protein that inhibits the enzymes that break down blood clots. solutions to cover large incisions or stop heavy bleeding. The process involves the combination of the thrombin and fibrinogen whereby a clot develops in the same way that it would form during normal blood clotting through a series of chemical reactions known as the coagulation cascade. At the end of the cascade, the thrombin breaks up the fibrinogen molecules into smaller segments of a second blood protein called fibrin. The fibrin molecules arrange themselves into strands that are then cross-linked by a blood factor known as Factor XIII to form a lattice or net-like pattern that stabilizes the clot.

The nose pad cushion can be formed where by a water vapor-permeable polyurethane film backing layer provides a self adherent border together with an absorbent for providing a moist wound environment. An adsorbent adhesive film dressing can also be applied to the nose pad cushion as a backing.

Thus, the nose pad cushion of the present invention uses a layer of absorbent fibrous material which could be formed in batting sandwiched between layers of silicone, a gauze material covers the skin side of the pad and a vapor permeable polyurethane film backing layer covers the silicone layer on the mask side, a fibrin glue is used to adhere the gauze to the skin while a stronger removable adhesive adheres to the silicone layer or a polyurethane film covering the silicone layer to the mask. A clear peel-off film may be used to cover the fibrin glue and mask adhesive prior to use.

For example, FIG. 1 show's a nose cushioning pad 45. An inner layer is formed of a fibrous, foam or other porous absorbent material such as a soft gauze material 60 sandwiched between layers of a soft silicone material. The rear surface 62 of the silicone layer 59 is provided with a removable adhesive 63 to securely hold pad 45 to the upper nose area 30 of a face mask 25. The adhesive 63 is selected from a type that can easily allow the pad 45 to be pealed off of the mask 25 and disposed of so that another pad 45 can be applied in its place. The pad 45 may be pealed off of a face mask to which it was applied without leaving a substantial residue which would encumber the application of a replacement pad. In other words, it would leave the surface free of anything except, perhaps, a small and almost undetectable film of adhesive. A thin plastic film 64 can be used to cover the adhesive can be peeled off prior to use. The soft gauze material 60 is covered in the front and optionally on the sides by the front layer 50 of soft silicone material 59. The silicone material which removably adheres to dry skin can be formed containing a plurality of pores 55 which may contain medication which will transfer out onto a patients skin, if desired. SAFETAC is the brand name of a pliable adhesive silicone material useful in the present invention which is hydrophobic so that it will not stick to a moist wound but can stick to dry skin. MEPLIX BORDER and SAFETAC are trademarks of Molnlycke Health Care of Gamlestadsvagen in Goteborg, Sweden.

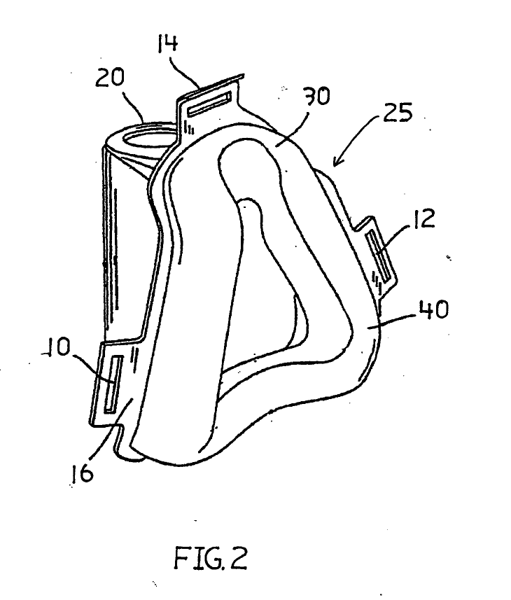

FIG. 2 show's a typical face mask 25 with strap loops 10, 12 and 14, frame 16, hose coupling 20, interface mask cushion surface 40 and upper nose interface surface area 30. The nose pad cushion 45 as shown in FIG. 1 is applied to the upper nose area 30 of face mask 25.

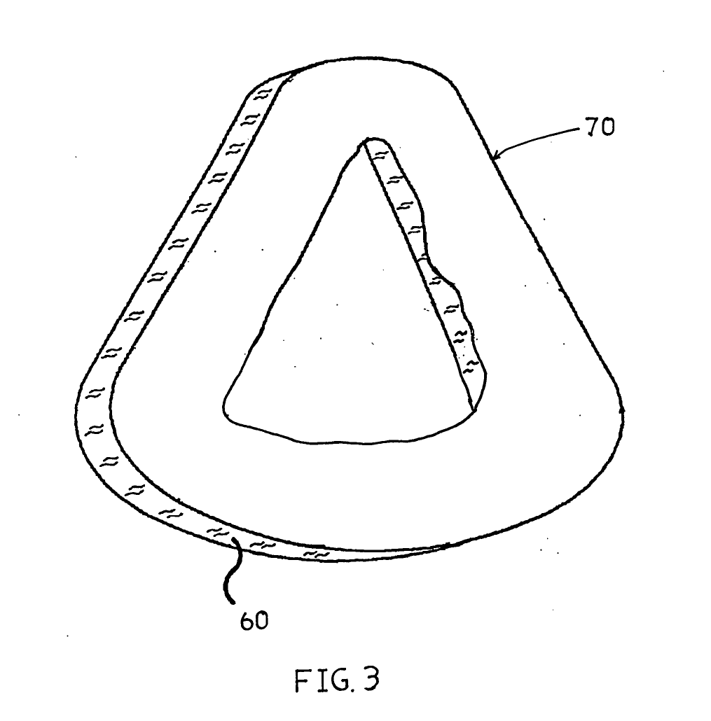

A further embodiment of the present invention is shown in FIG. 3, covers the entire cushion surface of a face mask 25 which normally contacts a user's skin. The nose pad cushion 70 as shown in FIG. 3 would be applied to the mask 25 cushion surface 40 extending around the entire periphery of the mask 25. Cushion 70 is shaped to cover the entire portion 40 of face mask 25 which would normally come into contact with a user's face which extends below the upper nose portion 30 and down over an area below the mouth of a user's face covering the upper lip area.

The NPPV peel-away medicated nose cushion pad is used as an adjunct for NPPV masks. The use of this pad prevents or treats nasal bridge pressure sores. In the acute care hospital setting, NPPV is used to support ventilation for the patient in respiratory failure. High pressures are used to increase the respiratory rate and the volume of air in each breath. Prolonged use of this therapy often causes pressure ulcers at the nasal bridge.

For example, the patient with no pressure sore, the pad can be used as a preventative measure. The padding is very thick to cushion the nasal bridge bone. The pad is disposable and it is recommended that it be changed every 72 hours. The pad adheres to the outside of the NPPV mask with adhesive that is peeled away for changing. The pad at the facial plane will adhere to the mask using a cryo precipitate gel. This plasma based fibrine gel dries quickly to create an antimicrobial seal. The skin condition will be monitored and documented using a pressure sore scale every 12 hours. The pad can be changed according to the needs of the patient. The patient diagnosed with Methicillin resistant Staph Aureous (MRSA) would have an antibiotic wound gel that is not methicillin based. For the patient with skin breakdown, a medicated pad should be used. The skin side of the pad or the fibrous portion is impregnated with one or a mixture of cryo precipitate fibrin gel with the following: lidocaine for pain relief, dexamethosone sodium phospate or hydrocortisone for anti-inflammatory relief, antibacterial/antibiotic ointment, silver based wound gel, zinc oxide ointment for skin irritation.

The interval between changes is normally determined by the amount of exudate produced by the wound, but the dressing may be left in place for several days on clean non-infected wounds.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplification presented herein above. Rather, what is intended to be covered is within the spirit and scope of the appended claims.

I claim:

1. A face mask cushion comprising an inner layer comprising a soft absorbent material sandwiched between a rear silicone layer coated on with a removable adhesive adherable to a cushion of a mask, a front silicone layer adherable to the dry skin of a patient and capable of being removed from said face mask without tearing or leaving substantial residue on said face mask.

2. The face mask cushion defined in claim 1 wherein said front layer and said inner layer have formed therein pores capable of holding medication to be transferred from said pores to said moist wound bed.

3. The face mask cushion defined in claim 2 wherein medication is present in said pores of said front layer and said inner layer.

4. The face mask cushion defined in claim 1 wherein said front layer has on its surface an adhesive which will adhere said cushion to dry skin, is incapable of sticking to a moist wound bed, and is capable of being easily removed from said dry skin without stress to the user.

5. A face mask cushion comprising:
a front soft silicone layer, said layer shaped to conform to a surface of the face of a user which would normally be in contact with said face mask, and incapable of sticking to a moist wound bed which is often present on a user's face;
an inner layer comprising a soft absorbent material; and
a rear adhesive layer, said rear layer shaped to conform to the area of said face mask which normally contacts the face of a user and said rear layer capable of being removed from a face mask without tearing or leaving substantial residue on said face mask.

6. The face mask cushion defined in claim 5 wherein said front layer and said inner layer have formed therein pores capable of holding medication to be transferred from said pores to said moist wound bed.

7. The face mask cushion defined in claim 6 wherein medication is present in said pores of said front layer and said inner layer.

8. The face mask cushion defined in claim 5 wherein said front layer has on its surface an adhesive which will adhere said cushion to dry skin, is incapable of sticking to a moist wound bed, and is capable of being easily removed from said dry skin without stress to the user.
